(19) United States
(12) Patent Application Publication
(10) Pub. No.: US 2022/0031738 A1
(43) Pub. Date: Feb. 3, 2022

(54) IRON FORMULATIONS FOR TOPICAL ADMINISTRATION AND METHODS OF TREATMENT OF IRON DEFICIENCY

(71) Applicant: DYVE BIOSCIENCES, INC., Thousand Oaks, CA (US)

(72) Inventors: Ryan Beal, Thousand Oaks, CA (US); Luke Gonzales, Thousand Oaks, CA (US); Kilmar Martinez, Thousand Oaks, CA (US); Brandon Sand, Thousand Oaks, CA (US); Lisa Misell, Thousand Oaks, CA (US); Nathan Fitzsimmons, Thousand Oaks, CA (US)

(73) Assignee: DYVE BIOSCIENCES, INC., Thousand Oaks, CA (US)

(21) Appl. No.: 17/282,991

(22) PCT Filed: Oct. 5, 2019
(86) PCT No.: PCT/US2019/054903

§ 371 Date: Apr. 5, 2021

Related U.S. Application Data
(60) Provisional application No. 62/742,180, filed on Oct. 5, 2018.

(51) Int. Cl. A61K 33/26; A61K 9/00; A61K 47/10; A61K 47/12; A61K 47/24; A61K 47/44; A61K 47/06; A61K 47/22; A61K 47/54; A61K 47/02; A61K 47/40; A61P 3/02

(52) U.S. Cl. CPC A61K 33/26; A61K 9/0014; A61K 47/10; A61K 47/12; A61K 47/24; A61P 3/02; A61K 47/06; A61K 47/22; A61K 47/547; A61K 47/02; A61K 47/40; A61K 47/44

(57) ABSTRACT

Provided herein are formulations for the transdermal administration of iron or an iron containing compound. Also provided are formulations that include iron chelators and antioxidants, and methods of using the formulations provided herein for the treatment of diseases and disorders relating to iron deficiency, anemia, and conditions associated with anemia.

### CROSS-REFERENCE TO RELATED APPLICATIONS

This application is the United States National Stage application of PCT/US2019/054903, filed Oct. 5, 2019, and claims the benefit of U.S. Provisional Application Ser. No. 62/742,180, filed Oct. 5, 2018, all incorporated by reference in their entirety herein.

### FIELD OF INVENTION

This invention relates generally to topical formulations comprising therapeutic agents, and in particular enhanced iron containing formulations suitable for transdermal or topical delivery and methods of treating iron deficiency and related disorders.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Iron deficiency, or sideropaenia, is the state in which a body has inadequate iron to supply its needs. Iron is present in all cells in the human body and has several vital functions, most notably carrying oxygen to the tissues from the lungs as a key component of the hemoglobin protein, acting as a transport medium for electrons within the cells in the form of cytochromes, and facilitating oxygen enzyme reactions in various tissues. Too little iron can interfere with these vital functions and lead to morbidity and death.

Total body iron averages approximately 3.8 g in men and 2.3 g in women. In blood plasma, iron is carried tightly bound to the protein transferrin. There are several mechanisms that control human iron metabolism and safeguard against iron deficiency. The main regulatory mechanism is situated in the gastrointestinal tract.

Iron exists either in the free form or heme iron in the diet. The free iron from the diet is converted from ferric form to ferrous form in the intestinal lumen and gets transported into the enterocyte cells. Iron can be stored in intestinal enterocytes bound to ferritin or transported to blood. Once it enters the systemic circulation, iron binds to the serum protein, transferrin. Transferrin is responsible for the transport of iron and carries iron to bone marrow for hemoglobin synthesis and other tissues in the body. Iron homeostasis in the system is closely regulated as there is no physiological mechanism for iron excretion from the body. Excess iron accumulation in the system causes organ dysfunction by producing the reactive oxygen species (ROS) through the Fenton reaction. ROS production can lead to a number of oxidative challenges throughout the body.

Iron must be supplied by the diet. When loss of iron is not sufficiently compensated by adequate intake of iron from the diet, a state of iron deficiency develops. A U.S. federal survey of food consumption determined that for women and men over the age of 19, average consumption from foods and beverages was 13.1 and 18.0 mg/day, respectively. For women, 16% in the age range 14-50 years consumed less than the Estimated Average Requirement (EAR), for men ages 19 and up, fewer than 3%. When a state of under consumption and iron deficiency is prolonged and uncorrected, it leads to iron deficiency anemia.

Iron deficiency anemia is one of the major nutritional deficiency disorders affecting over 1.5 B people worldwide. Iron deficiency anemia occurs due to decreased absorption of iron from diet, chronic blood loss and other associated diseases. Women and children are most commonly affected as are vegetarians and frequent blood donors. Sources of blood loss can include heavy periods, childbirth, uterine fibroids, stomach ulcers, colon cancer, inflammatory bowel disease, over-exertion in athletics, and urinary tract bleeding. A poor ability to absorb iron may occur as a result of Crohn's disease or a gastric bypass. In the developing world, parasitic worms, malaria, and HIV/AIDS increase the risk.

Without enough iron the body cannot produce enough hemoglobin. This leads to a number of symptoms including extreme fatigue, weakness, confusion, pale skin, chest pain, headache, cold hands and feet, brittle nails, poor appetite, and growth and developmental delays in children.

Diagnosis is typically done via blood analysis assessing red blood cell size and color, hematocrit levels, hemoglobin levels, and ferritin levels. Additional test to identify sources of blood loss can also include endoscopy, colonoscopy, and ultrasound.

Current treatments for iron deficiency are primarily diet modification and iron supplementation. Diet modification involves adding iron-rich foods like red meat, seafood, beans, dark leafy green vegetables, dried fruit, iron-fortified cereals, etc. Foods rich in vitamin C are also known to increase absorption from the gastrointestinal tract.

Iron supplementation with iron tablets or liquid form is often recommended. Iron supplements are highly recommended by doctors during pregnancy to avoid the birth of low weight child and prenatal complications. Iron supplements are used in various applications such as additional supplements, sports nutrition and medicinal supplements, taken orally or injected. Parenteral iron therapy is recommended only in severe iron deficiency conditions.

While oral iron supplementation plays an extremely important role in overall wellness, its effectiveness is limited by low bioavailability and poor tolerability. In addition, oral iron supplementation is known to cause a number of adverse side effects including diarrhea, constipation and abdominal cramping/pain. In addition, the unpleasant taste and odor of iron salts results in poor patient adherence, particularly among pregnant women and children. The various causes of poor tolerability lead to poor compliance with therapy, which has consequences for the efficacy of treatment.

In addition, the taking of oral iron supplements in excess of 200 mg/day causes a relative overabundance of iron that can alter the types of bacteria that are present within the gut. There have been concerns regarding parenteral iron being administered whilst bacteremia is present, although this has not been borne out in clinical practice. A moderate iron deficiency, in contrast, can provide protection against acute infection, especially against organisms that reside within hepatocytes and macrophages, such as malaria and tuberculosis. This is mainly beneficial in regions with a high prevalence of these diseases and where standard treatment is unavailable.

Parenteral iron therapy is recommended only in severe iron deficiency conditions because of its invasiveness and systemic side effects due to colloidal nature of the parenteral iron products. Parenteral iron formulations that are approved for clinical use include ferric carboxymaltose, iron dextran (ID), ferumoxytol, iron sucrose and sodium ferric gluconate. The iron overload with parenteral formulations was reported to cause anaphylactic reactions and rarely even mortality.

With all the disadvantages associated with oral and parenteral delivery of iron, there is a need for the development of an alternative and safe mode of administration of iron products. Accordingly, new treatments, formulations and methods of administration of formulations need to be developed that overcome the current deficiencies in such buffering formulations in order to determine the efficacy of various buffering formulations on the treatment of iron deficiency.

### SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

Iron is a critical component for numerous biologic functions including oxygen and electron transport and DNA and RNA synthesis. Iron deficiency is one of the most common nutritional deficiencies today and can lead to iron deficiency anemia which has numerous serious clinical consequences. Oral supplementation is the most common treatment today but is associated with poor bioavailability and severe GI distress which limit its adherence and efficacy.

Applicants have found that the drawbacks of intravenous and oral administration of iron supplementation can be overcome by administering iron or one or more iron containing compound topically and/or transdermally.

Topically delivered iron circumvents the GI challenges and provides a patient-friendly alternative to this condition. However, topically applied iron could also lead to iron accumulation in the dermis which could leave a patient vulnerable to photooxidative generation of reactive oxygen species (ROS) which is associated with a number of cutaneous pathologies such as skin cancer, photosensitization, and photoaging among others. This invention provides for a method of safe and effective topical delivery of iron. In addition to topically delivering iron systemically, this invention also includes iron chelators to minimize epidermal iron accumulation, and antioxidants to curtail ROS generation. These elements can be combined in a single use formulation or alternatively in separately applied formulations.

Accordingly, an aspect of the invention is to provide formulations for transdermal delivery of iron or one or more iron containing compound through the skin of a subject.

An exemplary embodiment of a formulation for transdermal delivery of iron or one or more iron containing compound through the skin of a subject comprises one or more iron containing compound in an amount of about 3.0% to about 40.0% w/w; Phospholipon® 90G in an amount from 0.0% to 15.0% w/w; cetyl alcohol in an amount from 0.0% to 5.0% w/w; stearic acid in an amount from 0.0% to 5.0% w/w; triacetin in an amount from 0.0% to 40.0% w/w; lecithin in an amount from 0.0% to 10.0% w/w; almond oil in an amount from 0.0% to 15.0% w/w; Cetiol® Ultimate in an amount from 0.0% to 20.0% w/w; Poloxamer 407 (e.g. Pluronic®) in an amount of from 0.0% to 20.0% w/w; glacial acetic acid in an amount from 0.0% to 10.0% w/w; propanoic acid in an amount from 0.0% to 5.0% w/w; cyclohexane in an amount from 0.0% to 10.0% w/w; tego 13-06 in an amount from 0.0% to 10.0% w/w; Durosoft® PK-SG in an amount from 0.0% to 3.0% w/w; HP beta CD in an amount from 0.0% to 1.0% w/w; citric acid in an amount from 0.0% to 5.0% w/w; vitamin C in an amount from 0.0% to about 5.0% w/w; vitamin E in an amount from 0.0% to about 5.0% w/w; an iron chelator (e.g., EDTA) in an amount from 0.0% to about 20.0% w/w; titanium dioxide in an amount from 0.0% to about 5.0% w/w; and water.

The iron or one or more iron containing compound can be ferrous sulfate or other iron containing compounds, salts, and the like.

The formulations may optionally contain an iron chelator, an antioxidant, or another active agent. Accordingly, one embodiment of a formulation for transdermal delivery of iron or one or more iron containing compound through the skin of a subject, comprises ferrous sulfate in an amount of about 3.0% to about 40.0% w/w; an iron chelator; an antioxidant; and optionally, one or more of the following: Phospholipon® 90G in an amount of about 5.0% w/w to about 15.0% w/w; cetyl alcohol in an amount of about 1.0% w/w to about 5.0% w/w; stearic acid in an amount of about 0.5% w/w to about 5.0% w/w; triacetin in an amount of about 5.0% w/w to about 25.0% w/w; lecithin in an amount of about 1.0% w/w to about 10.0% w/w; almond oil in an amount of about 3% w/w to about 15.0% w/w; Cetiol® Ultimate in an amount of about 3.0% w/w to about 20.0% w/w; Poloxamer 407 in an amount of about 2.0% w/w to about 20.0% w/w; glacial acetic acid in an amount of about 2.0% w/w to about 10.0% w/w; propanoic acid in an amount of about 1.0% w/w to about 5.0% w/w; cyclohexane in an amount of about 2.0% w/w to about 10.0% w/w; Tego 13-06 in an amount of about 2.0% w/w to about 10.0% w/w; Durosoft® PK-SG in an amount of about 0.5% w/w to about 3.0% w/w; HP beta CD in an amount up to about 1.0% w/w; citric acid in an amount up to about 5.0% w/w; titanium dioxide in an amount up to about 5.0% w/w; and water (H₂O) to complete. A formulation according this embodiment of the invention may comprise ferrous sulfate in an amount of about between about 3.0% w/w and about 15.0% w/w.

In another aspect, methods of treating an iron deficiency or related disorder are provided that comprise administering an effective amount of a formulation provided herein where the administration is effective to improve or treat the iron deficiency or related condition or disorder. Examples of iron an deficiency or related disorder include without limitation anemia, geriatric anemia or an anemia associated with aging, an anemia associated with sickle cell disease, an anemia associated with a bleeding disorder, an anemia associated with an iron deficiency, an anemia associated with a blood cancer, an anemia associated with a hematological disease or disorder, an anemia associated with cancer, an anemia associated with a blood cancer, supplementation of iron in non-hemodialysis patients with chronic kidney disease, progressive renal insufficiency, supplementation of iron in a pregnant woman having or susceptible to having anemia, anemia associated with inflammatory bowel disease, to supplement iron in iron depletion associated with athletic training in non-anemic women, and the supplementation of iron in anemic cancer patients also treated with darbepoetin alfa.

### DETAILED DESCRIPTION

The practices described herein employ, unless otherwise indicated, conventional techniques of tissue culture, immunology, molecular biology, microbiology, cell biology and recombinant DNA, which are within the skill of the art. See, e.g., Harlow and Lane eds. (1999) Antibodies, A Laboratory Manual and Herzenberg et al. eds (1996) Weir's Handbook of Experimental Immunology.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof. On the other hand “one” designates the singular.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation”, may or may not refer to a pharmaceutically acceptable composition for administration to humans or animals, and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

In an embodiment, a “pharmaceutical composition” is intended to include, without limitation, the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers, without limitation, to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will, without limitation, vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effected may, without necessarily being therapeutic, also be a cosmetic effect, in particular for treatment for disorders of the skin described herein.

In an embodiment, a “subject” of diagnosis or treatment is, without limitation, a prokaryotic or a eukaryotic cell, a tissue culture, a tissue or an animal, e.g. a mammal, including a human. Non-human animals subject to diagnosis or treatment include, for example, without limitation, a simian, a murine, a canine, a leporid, such as a rabbit, livestock, sport animals, and pets.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

### Formulations

To date, people have tried to drive penetration of various iron complexes using iontophoresis, electroporation, and microneedles. Passive transdermal delivery has not been successful. Transdermal delivery of iron could be a potential method of treating iron deficiency with advantages such as noninvasiveness, safety and patient compliance that this route could offer. Transdermal delivery of iron would be attractive to patients due to the fact that this route could overcome the potential gastric and systemic side effects associated with oral and parenteral delivery respectively.

The normal physiology of iron in the skin is complex and not clearly understood. It is known that iron levels in normal epidermis are thought to vary over a wide range (Molin and Wester, 1976; Kurz et al., 1987). Within normal dermis, iron levels also vary and are thought to increase during the aging process (Leveque et al., 2003). Furthermore, iron-containing proteins have specific function such as the metabolism of collagen by procollagen-proline dioxygenase (Richardson et al., 1996; Polefka et al., 2012; FIG. 1). Iron is not actively excreted from the body, however the skin is a key organ in iron hemostasis and nearly 25% of absorbed iron is normally eliminated from the body by exfoliation of epidermal cells (Weintraub et al, 1965; Jacob et al, 1981). Current theories regarding the underlying mechanisms of desquamation include active dissolution of desmosomes involved in keratinocyte cell-cell adhesion, by hydrolytic protease digestion (Milstone, 2004). Desquamation of keratinocytes is thought to account for 20-25% of absorbed iron that is lost (Jacob et al., 1981). Yet overall, the daily loss of iron by desquamation is approximately 25% that of daily urinary iron excretion (Molin and Wester, 1976).

Topical application of iron across mouse skin from water-in-oil and oil-in-water emulsion type ointments containing iron chelates has been reported in the past by Minato et al. In this study, Minato and colleagues (1967) evaluated the absorption of water-soluble substances from hydrophilic and absorption-based ointments using radioactive iron complexes with ethylenediaminetetraacetic acid and cupferron as model substance. The average absorption of iron from water-in-oil ointment containing water soluble iron-ethylenediaminetetraacetic acid complex was approximately 80% while it was only approximately 55% from oil-in-water ointment. These experiments were conducted with an ˜0.1% w/w formulation containing the radiolabeled iron-chelate. While absorption was high the starting concentrations were too low to offer a reasonable supplementation option.

As previously reported, in case of transdermal iron replenishment therapy, the choice of iron source has been challenging because all the iron salts used in oral iron therapy are hydrophilic and the products approved for parenteral therapy are large in size due to their colloidal nature. Moreover, the release of free iron from ionization of iron salts, in the systemic circulation is a major concern due to the risk of generation of reactive oxygen species (ROS) following oxidative stress at the cellular level.

The main causes of oxidative stress in the skin are reactive oxygen species (ROS) generated in the skin by ultraviolet (UVA) 320-400 nm portion of the UVA spectrum. Iron plays a key role in oxidative stress processes, as it is a transition metal, which exists in two stable states, Fe2+(electron donor) and Fe3+(electron acceptor). Intracellular labile iron can undergo redox cycling between its most stable oxidation states (Fe2+/Fe3+) and react with ROS such as superoxide anion, hydrogen peroxide, giving rise to hydroxyl radicals via the Fenton reaction or superoxide-driven Fenton chemistry (Pelle et al., 2011).

Exposure of skin fibroblasts to UVA can generate ROS that promote oxidative damage in lysosomal, mitrochondrial, nuclear, and plasma membranes. Ultimately loss of plasma membrane integrity together with mitrochondrial ATP depletion results in necrotic cell death (Aroun et al., 2012). It is thought that compared with skin fibroblasts, keratinocytes are more resistant to UVA mediated membrane damage and cytotoxicity. In vitro studies have shown that although UVA starts lysosomal damage, ferritin degradation and cytosolic labile iron release in keratinocytes, the absolute level of UVA induced labile iron release is several fold lower than in fibroblasts, suggesting a link between labile iron release and keratinocyte resistance to UVA mediated damage (Zhong et al., 2004).

Generation of the reactive oxygen species (ROS) in skin by exposure to ultraviolet (UV) radiation induces a number of cutaneous pathologies such as skin cancer, photosensitization, and photoaging among others. Skin iron catalyzes UV generation of ROS. Topical application of iron chelators reduces erythema, epidermal and dermal hypertrophy, wrinkle formation, tumor appearance. It has been proposed that iron chelators can be useful agents against damaging effects of both short- and long-term UV exposure.

Many types of iron and iron containing compounds are suitable for use in formulations for transdermal delivery of iron or one or more iron containing compound through the skin of a subject, including without limitation, sucrosomial iron, polysaccharide iron complex, ferrous fumarate, ferrous gluconate, ferrous sulfate, ferric carboxymaltose, ferumoxytol, iron isomaltoside 1000, ferric gluconate, iron sucrose, and ferric pyrophosphate.

A formulation for transdermal delivery of iron or one or more iron containing compound through the skin of a subject according to the invention may comprise the following:
Ferrous sulfate in an amount of about 3.0% to about 40.0% w/w;
Phospholipon® 90G in an amount of up to about 15.0% w/w;
Cetyl alcohol in an amount of up to about 5.0% w/w;
Stearic acid in an amount of up to about 5.0% w/w;
Triacetin in an amount of up to about 25.0% w/w;
Lecithin in an amount of up to about 10.0% w/w;
Almond Oil in an amount of up to about 15.0% w/w;
Cetiol® Ultimate in an amount of up to about 20.0% w/w;
Poloxamer 407 in an amount of about up about 20.0% w/w;
Glacial acetic acid in an amount of up to about 10.0% w/w;
Propanoic acid in an amount of up to about 5.0% w/w;
Cyclohexane in an amount of up to about 10.0% w/w;
Tego 13-06 in an amount of up to about 10.0% w/w;
Durosoft® PK-SG in an amount of up to about 3.0% w/w;
HP beta CD in an amount of up to about 1.0% w/w;
Citric acid in an amount of about 5.0% w/w;
Vitamin C in an amount of up about 5.0% w/w;
Vitamin E in an amount of up to about 5.0% w/w;
EDTA in an amount of up to about 20.0% w/w;
Titanium dioxide in an amount of up to about 5.0% w/w; and
H₂O to complete.

In some embodiments, the formulation comprises:
Ferrous sulfate in an amount of about 3.0% to about 15.0% w/w;
Phospholipon® 90G in an amount of about 5.0% w/w to about 15.0% w/w;
Cetyl alcohol in an amount of about 1.0% w/w to about 5.0% w/w;
Stearic acid in an amount of about 0.5% w/w to about 5.0% w/w;
Triacetin in an amount of about 5.0% w/w to about 25.0% w/w;
Lecithin in an amount of about 1.0% w/w to about 10.0% w/w;
Almond Oil in an amount of about 3% w/w to about 15.0% w/w;
Cetiol® Ultimate in an amount of about 3.0% w/w to about 20.0% w/w;
Poloxamer 407 in an amount of about 2.0% w/w to about 20.0% w/w;
Glacial acetic acid in an amount of about 2.0% w/w to about 10.0% w/w;
Propanoic acid in an amount of about 1.0% w/w to about 5.0% w/w;
Cyclohexane in an amount of about 2.0% w/w to about 10.0% w/w;
Tego 13-06 in an amount of about 2.0% w/w to about 10.0% w/w;
Durosoft® PK-SG in an amount of about 0.5% w/w to about 3.0% w/w;
HP beta CD in an amount of about 0.0% w/w to about 1.0% w/w;
Citric acid in an amount of about 0.0% w/w to about 5.0% w/w;
Vitamin C in an amount of about 0.0% w/w to about 5.0% w/w;
Vitamin E in an amount of about 0.0% w/w to about 5.0% w/w;
EDTA in an amount of about 1.0% w/w to about 20.0% w/w;
Titanium dioxide in an amount of about 0.0% w/w to about 5.0% w/w; and
H₂O to complete.

In a particular embodiment, the formulation comprises:
Ferrous sulfate in an amount of about 6.0% w/w;
Phospholipon® 90G in an amount of about 8.0% w/w;
Cetyl alcohol in an amount of about 3.0% w/w;
Stearic acid in an amount of about 2.0% w/w;
Triacetin in an amount of about 15% w/w;
Lecithin in an amount of about 4.0% w/w;
Almond Oil in an amount of about 12% w/w;
Cetiol® Ultimate in an amount of about 10.0% w/w;
Poloxamer 407 (Pluronic®) in an amount of about 5.0% w/w;
Glacial acetic acid in an amount of about 5.0% w/w;
Propanoic acid in an amount of about 2.0% w/w;
Cyclohexane in an amount of about 5.0% w/w;
Tego 13-06 in an amount of about 4.0% w/w;
Durosoft® PK-SG in an amount of about 1.0% w/w;
HP beta CD in an amount of about 0.1% w/w;
Citric acid in an amount of about 2.0% w/w;
Vitamin C in an amount of about 2.0% w/w;
Vitamin E in an amount of about 2.0% w/w;
EDTA in an amount of about 10.0% w/w;
Titanium dioxide in an amount of about 2.0% w/w; and
H₂O to complete.

In another aspect, a formulation for transdermal delivery of iron or one or more iron containing compound through the skin provided herein may combine an iron chelator in addition to iron or one or more iron containing compound. Accordingly, certain embodiments have one or more iron containing compound formulated with an iron chelator. One embodiment comprises ferrous sulfate in an amount of about 3.0% to about 40.0% w/w; an iron chelator; and optionally, one or more of the following: an antioxidant; Phospholipon® 90G in an amount of about 5.0% w/w to about 15.0% w/w; cetyl alcohol in an amount of about 1.0% w/w to about 5.0% w/w; stearic acid in an amount of about 0.5% w/w to about 5.0% w/w; triacetin in an amount of about 5.0% w/w to about 25.0% w/w; lecithin in an amount of about 1.0% w/w to about 10.0% w/w; almond oil in an amount of about 3% w/w to about 15.0% w/w; Cetiol® Ultimate in an amount of about 3.0% w/w to about 20.0% w/w; Poloxamer 407 in an amount of about 2.0% w/w to about 20.0% w/w; glacial acetic acid in an amount of about 2.0% w/w to about 10.0% w/w; propanoic acid in an amount of about 1.0% w/w to about 5.0% w/w; cyclohexane in an amount of about 2.0% w/w to about 10.0% w/w; Tego 13-06 in an amount of about 2.0% w/w to about 10.0% w/w; Durosoft® PK-SG in an amount of about 0.5% w/w to about 3.0% w/w; HP beta CD in an amount up to about 1.0% w/w; citric acid in an amount up to about 5.0% w/w; titanium dioxide in an amount up to about 5.0% w/w; and water (H₂O).

In another aspect, a formulation for transdermal delivery of iron or one or more iron containing compound through the skin provided herein may combine an antioxidant in addition to iron or one or more iron containing compound. Accordingly, certain embodiments have one or more iron containing compound formulated with an antioxidant. One embodiment comprises ferrous sulfate in an amount of about 3.0% to about 40.0% w/w; an antioxidant; and optionally, one or more of the following: an iron chelator; Phospholipon® 90G in an amount of about 5.0% w/w to about 15.0% w/w; cetyl alcohol in an amount of about 1.0% w/w to about 5.0% w/w; stearic acid in an amount of about 0.5% w/w to about 5.0% w/w; triacetin in an amount of about 5.0% w/w to about 25.0% w/w; lecithin in an amount of about 1.0% w/w to about 10.0% w/w; almond oil in an amount of about 3% w/w to about 15.0% w/w; Cetiol® Ultimate in an amount of about 3.0% w/w to about 20.0% w/w; Poloxamer 407 in an amount of about 2.0% w/w to about 20.0% w/w; glacial acetic acid in an amount of about 2.0% w/w to about 10.0% w/w; propanoic acid in an amount of about 1.0% w/w to about 5.0% w/w; cyclohexane in an amount of about 2.0% w/w to about 10.0% w/w; Tego 13-06 in an amount of about 2.0% w/w to about 10.0% w/w; Durosoft® PK-SG in an amount of about 0.5% w/w to about 3.0% w/w; HP beta CD in an amount up to about 1.0% w/w; citric acid in an amount up to about 5.0% w/w; titanium dioxide in an amount up to about 5.0% w/w; and water (H₂O).

Another exemplary embodiment comprises ferrous sulfate in an amount of about 3.0% to about 40.0% w/w; an iron chelator; an antioxidant; and optionally, one or more of the following: Phospholipon® 90G in an amount of about 5.0% w/w to about 15.0% w/w; cetyl alcohol in an amount of about 1.0% w/w to about 5.0% w/w; stearic acid in an amount of about 0.5% w/w to about 5.0% w/w; triacetin in an amount of about 5.0% w/w to about 25.0% w/w; lecithin in an amount of about 1.0% w/w to about 10.0% w/w; almond oil in an amount of about 3% w/w to about 15.0% w/w; Cetiol® Ultimate in an amount of about 3.0% w/w to about 20.0% w/w; Poloxamer 407 in an amount of about 2.0% w/w to about 20.0% w/w; glacial acetic acid in an amount of about 2.0% w/w to about 10.0% w/w; propanoic acid in an amount of about 1.0% w/w to about 5.0% w/w; cyclohexane in an amount of about 2.0% w/w to about 10.0% w/w; Tego 13-06 in an amount of about 2.0% w/w to about 10.0% w/w; Durosoft® PK-SG in an amount of about 0.5% w/w to about 3.0% w/w; HP beta CD in an amount up to about 1.0% w/w; citric acid in an amount up to about 5.0% w/w; titanium dioxide in an amount up to about 5.0% w/w; and water (H₂O).

In another aspect, a formulation for transdermal delivery of iron or one or more iron containing compound through the skin provided herein may combine an antioxidant in addition to iron or one or more iron containing compound. Accordingly, certain embodiments have one or more iron containing compound formulated with an antioxidant.

Accordingly, in some embodiments, the formulation comprises:
ferrous sulfate in an amount of about 3.0% to about 40.0% w/w;
an iron chelator;
an antioxidant;
and optionally, one or more of the following:
Phospholipon® 90G in an amount of about 5.0% w/w to about 15.0% w/w;
cetyl alcohol in an amount of about 1.0% w/w to about 5.0% w/w;
stearic acid in an amount of about 0.5% w/w to about 5.0% w/w;
triacetin in an amount of about 5.0% w/w to about 25.0% w/w;
lecithin in an amount of about 1.0% w/w to about 10.0% w/w;
almond oil in an amount of about 3% w/w to about 15.0% w/w;
Cetiol® Ultimate in an amount of about 3.0% w/w to about 20.0% w/w;
Poloxamer 407 in an amount of about 2.0% w/w to about 20.0% w/w;
glacial acetic acid in an amount of about 2.0% w/w to about 10.0% w/w;
propanoic acid in an amount of about 1.0% w/w to about 5.0% w/w;
cyclohexane in an amount of about 2.0% w/w to about 10.0% w/w;
Tego 13-06 in an amount of about 2.0% w/w to about 10.0% w/w;
Durosoft® PK-SG in an amount of about 0.5% w/w to about 3.0% w/w;
HP beta CD in an amount up to about 1.0% w/w;
citric acid in an amount up to about 5.0% w/w;
titanium dioxide in an amount up to about 5.0% w/w; and
H₂O to complete.

A single formulation provided herein may comprise any combination of the following three components i) topically applied iron (e.g. iron sulfate), ii) a topically applies iron chelator, and iii) a topically applied antioxidant. Additionally, one or more of the above i)-iii) may be formulated separately or in any combination and applied separately as a topical formulation. Additionally, the formulations could be applied sequentially or alternatively at various times through the day (e.g., iron and/or antioxidants applied at night, iron chelators and/or antioxidants applied in the morning).

Formulations containing iron may be formulated at acidic pH to minimize the spontaneous oxidation Fe(II) into Fe(III).

Suitable nonlimiting exemplary iron chelators include deferoxamine, ethylenediaminetetraacetic acid (EDTA), 1,2-diethyl-3-hydroxypyridin-4-one (CP94), Desferol, Deferiprone and Deferasirox, succimer, trientine, Desferrithiocin, Clioquinol, O-trensox, Tachpyr, Dexrazoxane, Triapine, Pyridoxal isonicotinoyl hydrazone, Di-2-pyridylketone thiosemicarbazone series, Flavan-3-ol, Curcumin, Apocynin, Kolaviron, Floranol, Baicalein, Baicalin, ligustrazine, Quercetin, Epigallocatechin gallate, Theaflavin, Phytic acid, and Genistein.

Suitable nonlimiting exemplary antioxidants include glutathione, vitamin C, vitamin E, superoxide dismutase, catalase, pNaKtide, Butylated hydroxytoluene, Butylated hydroxyanisole, tert-Butylhydroquinone, HP beta CD, resveratrol, retinol, coenzyme q10, niacinamide, polyphenols, flavenoids, beta-carotene, lutein, and lycopene.

Certain embodiments of formulations provided herein may be supplemented with formulation components described in greater detail in the inventor's related applications, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

The formulations comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

For topical administration, and in particular transdermal administration, the formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. Particularly suitable penetrants especially for those that contain at least one agent other than buffer include those that are described in the US2009/0053290, WO2014/209910, and WO2017/127834, incorporated by reference herein. In addition to formulations with penetrants, transdermal delivery can be affected by mechanically disrupting the surface of the skin to encourage penetration, or simply by supplying the formulation applied to the skin under an occlusive patch.

Alternatively, the penetrant portion comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Lecithin organogel is a combination of lecithin with a gelling component, which is typically amphiphilic. Suitable gelling components also include isopropyl palmitate, ethyl laurate, ethyl myristate and isopropyl myristate. In some embodiments, the formulation comprises a gelling agent in an amount less than 5% w/w of the formulation. Certain hydrocarbons, such as cyclopentane, cyclooctane, trans-decalin, trans-pinane, n-pentane, n-hexane, n-hexadecane may also be used. Thus, an important permeation agent is a lecithin organogel, wherein the combination resulting from lecithin and the organic solvent acts as a permeation agent. In some embodiments, the penetrant portion comprises lecithin organogel, an alcohol, a surfactant, and a polar solvent. In some embodiments, the lecithin organogel is a combination of soy lecithin and isopropyl palmitate. In some embodiments, the penetrant portion comprises lecithin and isopropyl palmitate, undecane, isododecane, isopropyl stearate, or a combination thereof. In some embodiments, the formulation comprises Lipmax™ (sold by Lucas Meyer Cosmetics) in an amount between about 1-20% w/w or an equivalent 50/50 mixture of isopropyl palmitate and lecithin. Lecithin organogels are clear, thermodynamically stable, viscoelastic, and biocompatible jelly-like phases composed of hydrated phospholipids and appropriate organic liquid. An example of a suitable lecithin organogel is lecithin isopropyl palmitate, which is formed when isopropyl palmitate is used to dissolve lecithin. The ratio of lecithin to isopropyl palmitate may be 50:50. Illustrated below in the Examples is a formulation containing soy lecithin in combination with isopropyl palmitate; however, other lecithins could also be used such as egg lecithin or synthetic lecithins. Various esters of long chain fatty acids may also be included. Methods for making such lecithin organogels are well known in the art. In most embodiments, the lecithin organogel is present in the final formulation is less than about 20% w/w. In those compositions used to dissolve fat deposits, to alleviate pain from fat removal or in anhydrous compositions, the concentration of lecithin organogel may be as low as 0.5% w/w, 1% w/w, 5% w/w, 10% w/w or 20% w/w. In some embodiments, the penetrant portion comprises a mixture of xanthan gum, lecithin, sclerotium gum, pullulan, or a combination thereof in an amount less than 2% w/w, 5% w/w, or 10% w/w of the formulation. In some embodiments, the formulation comprises Siligel™ in an amount between about 1-5 w/w or 5-15% w/w, or an equivalent mixture of xanthan gum, lecithin, sclerotium gum, and pullulan. In some embodiments, the penetrant portion comprises a mixture of caprylic triglycerides and capric triglycerides in amount less than 2% w/w, 8% w/w, or 10% w/w of the formulation. In some embodiments, the formulation comprises Myritol® 312 in an amount between about 0.5-10% w/w, or an equivalent mixture of caprylic triglycerides and capric triglycerides.

In some embodiments, the penetrant portion comprises phosphatidyl choline in amount less than 12% w/w or 18% w/w of the formulation. In some embodiments, the penetrant portion comprises a phospholipid in amount less than 12% w/w or 18% w/w of the formulation. In some embodiments, the penetrant portion comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the penetrant portion comprises cetyl alcohol in amount less than 2% w/w, 5% w/w, or 8% w/w of the formulation. In some embodiments, the penetrant portion comprises benzyl alcohol in an amount less than about 2% w/w, 5% w/w, or 8% w/w. In some embodiments, the penetrant portion comprises stearic acid in an amount less than 2% w/w, 5% w/w, or 8% w/w of the formulation.

Lecithin organogels may be in the form of vesicles, microemulsions and micellar systems. In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semi-solid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine.

The lecithin organogels are formulated with an additional component to assist in the formation of micelles or vascular structures. In one approach, the organogels are formulated with a polar component such as water, glycerol, ethyleneglycol or formamide, in particular with water. In general, a nonionic detergent such as a poloxamer in aqueous solution is used to top off. Alternatively, an anhydrous composition may be obtained by using, instead of a polar component, a material such as a bile salt. When formulated with bile salts, the mi cellular nature of the composition is altered so that rather than a more or less spherical vesicular form, the vesicles become wormlike and are able to accommodate larger guest molecules, as well as penetrate the epidermis more effectively. Suitable bile salts include salts of deoxycholic acid, taurocholic acid, glycocholic acid, taurochenodeoxycholic acid, glycochenodeoxycholic acid, cholic acid and the like. Certain detergents, such as Tween® 80 or Span® 80 may be used as alternatives. The percentage of these components in the anhydrous forms of the composition is in the range of 1% w/w-15% w/w. In some embodiments, the range of bile salt content is 2%-6% w/w or 1%-3.5% w/w. In these essentially anhydrous forms, powdered or micronized nonionic detergent is used to top off, typically in amounts of 20%-60% w/w. In one approach to determine the amount of bile salt, the % is calculated by dividing the % w/w of lecithin by 10.

An additional component in the formulations of the disclosure is an alcohol. Benzyl alcohol and ethanol are illustrated in the Examples. in particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are incltided. Due to the aromatic group present in a permeation enhancement formulation such as benzyl alcohol, the molecule has a polar end (the alcohol end) and a non-polar end (the benzene end). This enables the agent to dissolve a wider variety of drugs and agents. The alcohol concentration is substantially lower than the concentration of the lecithin organogel in the composition.

In some embodiments, as noted above, the performance of the formulations is further improved by including a nonionic detergent and polar gelling agent or including bile salts and a powdered surfactant. In both aqueous and anhydrous forms of the composition, detergents, typically nonionic detergents are added. In general, the nonionic detergent should be present in an amount of at least 2% w/w to 60% w/w. Typically, in the compositions wherein the formulation is topped off with a polar or aqueous solution containing detergent, the amount of detergent is relatively low—e.g., 2%-25% w/w, or 5-15% w/w or 7-12% w/w. However, in compositions comprising bile salts that are essentially anhydrous and are topped-off by powdered detergent, relatively higher percentages are usually used—e.g., 20%-60% w/w.

In some embodiments, the nonionic detergent provides suitable handling properties whereby the formulations are gel-like or creams at room temperature. To exert this effect, the detergent, typically a poloxamer, is present in an amount between about 2-12% w/w, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as Poloxamer 407 (e.g. Pluronic®) and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, the formulation also contains surfactant, typically, nonionic surfactant at 2-25% w/w along with a polar solvent wherein the polar solvent is present in an amount at least in molar excess of the nonionic surfactant. In these embodiments, typically, the composition comprises the above-referenced amounts of lecithin organogel and benzyl alcohol along with a carbonate salt with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount between about 2-25% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethyleneglycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Typical components that may also be included in the formulations are fatty acids, terpenes, lipids, and cationic, and anionic detergents. In some embodiments, the formulation further comprises tranexamic acid in an amount less than 2% w/w, 5% w/w, or 10% w/w of the formulation. In some embodiments, the formulation further comprises a polar solvent in an amount less than 2% w/w, 5% w/w, 10% w/w, or 20% w/w of the formulation. In some embodiments, the formulation further comprises a humectant, an emulsifier, an emollient, or a combination thereof. In some embodiments, the formulation further comprises ethylene glycol tetraacetic acid in an amount less than about 2% w/w, 5% w/w, or 10% w/w. In some embodiments, the formulation further comprises almond oil in an amount less than about 5% w/w. In some embodiments, the formulation further comprises a mixture of thermoplastic polyurethane and polycarbonate in an amount less than about 5% w/w. In some embodiments, the formulation further comprises phosphatidylethanolamine in an amount less than about 5 w/w. In some embodiments, the formulation further comprises an inositol phosphatide in an amount less than about 5% w/w.

Other solvents and related compounds that may be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C3-C6 triols or their mixtures and a polar lipid compound selected from C16 or C18 monounsaturated alcohol, C16 or C18 branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N,N-dimethylamino acetate. Examples of suitable fatty acid. include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that may be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants may be included, such as vitamin C, vitamin E, proanthocyanidin and a-lipoic acid typically in concentrations of 0.1%-2.5% w/w.

In some embodiments, it is desirable to adjust the pH of the formulation to assist in permeation or to adjust the nature of the iron containing compounds and/or other target compounds in the subject. In some instances, the pH is adjusted to a level of pH 9-11 or 10-11 which can be done by providing appropriate buffers or simply adjusting the pH with base. In other embodiments, it is desirable to adjust the pH of the formulation to reduce or prevent oxidation of iron, assist in permeation, or to adjust the nature of the iron containing compounds and/or of other target compounds in the subject to a level of pH 4-6, which can be done by providing appropriate buffers or simply adjusting the pH with an acid.

In some applications a formulation for transdermal delivery may, for example, comprise: Aveeno®, for example in an amount between about 10-95% w/w; between about 20-85% w/w, between about 20-75% w/w, between about 20-50% w/w.

In some particular applications, in particular when administering the iron containing compound with another therapeutic agent, it may be desirable to further include an anesthetic, epinephrine or an alternate vasoconstrictor, such as phenylephrine or epinephrine sulfate may be included in the formulation if a stabilizing agent is present.

In any of the anesthetic compositions, it may be desirable to administer the epinephrine in tandem with the transdermal anesthetic. Alternatively, treatment of the epinephrine with a chelator, such as the iron chelator Desferal® may stabilize the epinephrine sufficiently to include it in the transdermal formulation.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included and the packaging also can include written or web-accessible instructions for using the formulation. A container can include, for example, a vial, bottle, patch, syringe, pre-filled syringe, tube or any of a variety of formats well known in the art for multi-dispenser packaging.

### Administration and Dosing

The formulations provided herein can be topically administered in any form. For administration for the treatment of skin conditions a sufficient amount of the topical composition can be applied onto a desired area and surrounding skin, for example, in an amount sufficient to cover a desired skin surface. The formulations can be applied to any skin surface, including for example, facial skin, and the skin of the hands, neck, chest and/or scalp.

In applying the formulations of the invention, the formulation itself is simply placed on the skin and spread across the surface and/or massaged to aid in penetration. The amount of formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of the formulation to the skin and prevents distortion of the formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

Accordingly, in certain embodiments alternative methods of administering one or more buffering agent, therapeutic compounds, agents, drugs through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

It has been found, generally, that the requirements for effective penetration of the skin in the case of buffers as active agents are less restrictive than those required for alternative agents useful in preventing cancer metastasis. In addition, although for these indications' delivery to the locus of the solid tumor, including melanoma, or melasma or gout is desirable, effective systemic pH alteration can be used as a way to diagnose the effectiveness of penetration when topical administration is employed.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of the formulation itself. The application of the formulation may be by simple massaging onto the skin or by use of devices such as syringes or pumps. Patches could also be used. In some cases, it is helpful to cover the area of application to prevent evaporation or loss of the formulation.

Where the application area is essentially skin, it is helpful to seal-off the area of application subsequent to supplying the formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In some embodiments, the disclosure is directed to administering a therapeutic agent in combination with an iron containing compound or formulation provided herein. A wide variety of therapeutic agents may be used in the formulations or compositions and formulations for other routes of administration, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply affect transport across the skin into a localized subdermal location, such as treatment of nail fungus or modulation of hair growth or may affect systemic delivery such as is desirable in some instances where vaccines are used.

In addition to the compositions and formulations of the invention per se, the methods may employ a subsequent treatment with linoleic acid. As transdermal treatments generally open up the skin barrier, which is, indeed, their purpose, it is useful to seal the area of application after the treatment is finished. Thus, treatment with the formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow carbonates to pass through the epidermis to the dermis at least, and, if systemic administration is achieved, through the dermis itself.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

The formulations can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, for 4 weeks, or for 4 weeks or more. In some instances, it may also be desirable to continue treatment indefinitely for example to inhibit or prevent carcinogenesis or for improving, extending the duration of remission, or maintaining remission of a cancer or another disease or disorder. A suitable administration for a formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the topical formulation may be prepared. Single dose, unit dose, and once-daily disposable containers of the topical formulation are also provided.

The present topical formulation remains stable in storage for periods including up to about 5 years, between about 3 months and about 5 years, between about 3 months and about 4 years, between about 3 months and about 3 years, and alternately any time period between about 6 months and about 3 years.

A topical formulation described herein remains stable for up to at least 3 years at a temperature of less than or equal to 40° C. In an embodiment, the presently described topical formulation remains stable for at least 2 years at a temperature of less than or equal to 40° C. In an embodiment, the presently described formulation or emulsion remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described biocompatible composition in accordance with the subject matter described herein remains stable for an extended period of time when packaged in a multi-use container such as a bottle dispenser or the like, and exhibits equal to or even greater stability when packaged in a single-use package.

A suitable dose of iron or an iron containing compound administered topically as a formulation for subject (e.g. a human patient) is at least about 500 mg, at least about 750 mg, at least about 1000 mg, at least about 1.5 g, at least about 2.0 g, at least about 2.5 g, at least about 3.0 g, at least about 3.5 g, at least about 4.0 g, at least about 4.5 g, at least about 5.0 g, at least about 6.0 g, at least about 7.0 g, at least about 8.0 g, at least about 9.0 g, at least about 10.0 g, at least about 11 g, at least about 12 g, at least about 13 g, at least about 14 g, at least about 15 g, at least about 20 g, at least about 25 g, at least about 30 g, at least about 35 g, at least about 40 g, at least about 45 g, at least about 50 g, or more. This does is typically administered daily, twice a day, or three times a day, but it may also be administered four times a day, five times a day, or more than five times a day.

Alternatively, a suitable daily dose of iron or an iron containing compound administered topically as a formulation for subject is at least about 10 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, up to at least about 500 mg/kg or more.

In another aspect, a suitable daily dose of iron or an iron containing compound administered topically as a formulation for subject is about 10 mg/kg to about 1.0 g/kg, and more typically the daily dose is about 10 mg/kg to about 500 mg/kg, about 25 mg/kg to about 500 mg/kg, about 50 mg/kg to about 300 mg/kg, about 75 mg/kg to about 300 mg/kg, about 75 mg/kg to about 250 mg/kg, about 100 mg/kg to about 300 mg/kg, about 75 mg/kg to about 200 mg/kg, about 100 mg/kg to about 200 mg/kg, or alternative ranges.

Aspects of the present specification disclose that the symptoms associated with a disease or disorder described herein are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

The formulations as described herein can be used in the manufacture of medicaments and for the treatment of humans and other animals by administration in accordance with conventional procedures.

### EXPERIMENTAL EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

The following examples are intended to illustrate but not to limit the invention.

### Example 1—Use of Topically Delivered Iron Supplementation in Patients with Progressive Renal Insufficiency Treated with Erythropoietin

In this experiment, iron (in the form of ferrous sulfate) in formulations of the invention was tested for its ability to treat anemia in patients with progressive renal insufficiency, and compared to orally or intravenously delivered iron supplementation.

In vivo tests were performed as follows: Anemic human patients with progressive renal insufficiency were treated with either topical, oral or intravenous iron supplementation and evaluated at baseline, 3 months and 6 months. Trial design was built to assess these primary outcomes: hemoglobin response, serum ferritin levels, changes in red cell hypochromasia, and incidence of adverse events. Treatment groups randomized as follows:
Oral iron supplement group (ferrous sulphate 200 mg tds)
Intravenous iron supplement group (300 mg iron sucrose monthly)
Topical iron supplement group (3.34 g of topical formulation applied tds) Formulations detailed below in Table 1
In all groups Erythropoietin (rHuEpo) treatment was simultaneously commenced.

TABLE 1
Iron Formulation

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate | 6.00% |
| Total | 100.00% |

All treatment groups showed no statistical differences in hemoglobin response or changes in red cell hypochromasia. Serum ferritin levels were statistical significantly higher in the topical and intravenous treatment groups versus oral, but were not statistically different from each other.

Patients in the oral group reported more mild gastrointestinal symptoms but not enough to discontinue usage. There were allergic reactions reported in 10% of intravenous patients though none were reported for oral or transdermal patients.

### Example 2—Use of Topically Delivered Iron Supplementation for Treatment of Iron Deficiency in Non-Hemodialysis-Dependent Patients with Chronic Kidney Disease

In this experiment, iron (in the form of ferrous sulfate) in formulations of the invention was tested for its ability to treat iron deficiency in non-hemodialysis dependent patients with chronic kidney disease, and compared to orally or intravenously delivered iron supplementation.

In vivo tests were performed as follows: Anemic human patients with progressive renal insufficiency were treated with either topical, oral or intravenous iron supplementation. Trial design was built to assess these primary outcomes: hemoglobin response, and incidence of adverse events. Treatment groups randomized as follows:
a. Oral iron supplement group (ferrous sulphate 325 mg thrice daily for a total of 195 mg elemental iron daily)
b. Intravenous iron supplement group (intravenous ferric carboxymaltose 1000 mg over 15 min (with up to two additional doses of 500 mg at 2-week intervals))
c. Topical iron supplement group (5.42 g of topical formulation applied tds) Formulations detailed in Table 2 below.

TABLE 2
Iron Formulation

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate | 6.00% |
| Total | 100.00% |

The proportion of subjects achieving a hemoglobin increase ≥1 g/dL at any time was 60.4% in the intravenous group, 52.1% in the topical group and 34.7% in the oral group. Treatment-related adverse events were significantly fewer with ferric carboxymaltose and with topical iron than with oral iron (2.7% and 4.3% vs 26.2%).

### Example 3—Use of Topically Delivered Iron Supplementation for Treatment of Anemia in Pregnancy

In this experiment, iron (in the form of ferrous sulfate) in formulations of the invention was tested for its ability to treat anemia in pregnant patients, and compared to orally or intravenously delivered iron supplementation.

In vivo tests were performed as follows: Anemic pregnant human patients were treated with either topical, oral or intravenous iron supplementation during 14 to 34 weeks of pregnancy. Trial design was built to assess these primary outcomes: haemoglobin and serum ferritin levels. Treatment groups randomized as follows:
a. Oral iron supplement group (200 mg of ferrous fumarate per day)
b. Intravenous iron supplement group (200 mg of iron sucrose was administered in 100 ml 0.9% sodium chloride per day)
c. Topical iron supplement group (3.34 g of topical formulation applied per day) Formulations detailed in Table 3 below

TABLE 3
Iron Formulation

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate | 6.00% |
| Total | 100.00% |

The change in hemoglobin in pregnant women receiving intravenous iron was 22±11.5 g/L vs 15±10.5 g/L for topical iron and 12±9 g/L for oral iron. 55% participants in the intravenous group had an improvement in hemoglobin more than 20 g/L compared to only 16% of the topical group and 11% of the oral therapy group. 48% of patients in I.V group showed increase in ferritin level between 51 to 100 ng/ml in comparison to 35% of topical group and only 3.5% in oral group.

### Example 4—Use of Topically Delivered Iron Supplementation in Anemic Cancer Patients without Iron Deficiency Receiving Darbepoetin Alfa

In this experiment, iron (in the form of ferrous sulfate) in formulations of the invention was tested for its ability to help treat chemotherapy-induced anemia in cancer patients when combined with erythropoiesis-stimulating agents (ESAs), and compared to orally or intravenously delivered iron supplementation.

In vivo tests were performed as follows: human patients with chemotherapy-related anemia (Hb>8 g/dL<10 g/dL) and no absolute or functional iron deficiency were treated with darbepoetin alfa (500 mcg once every 3 weeks) and either topical, oral or intravenous iron supplementation for 8 weeks. Trial design was built to assess this primary outcome: Hb response as defined as the Hb increase ≥2 g/dL from baseline or the attainment Hb ≥12 g/dL. Treatment groups randomized as follows:
a. Oral iron supplement group (sucrosomial iron 30 mg daily)
b. Intravenous iron supplement group (ferric gluconate 125 mg weekly)
c. Topical iron supplement group (0.50 g of topical formulation applied daily) Formulations detailed in Table 4 below

TABLE 4
Iron Formulation

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate | 6.00% |
| Total | 100.00% |

There was no difference in the Hb response rate between the three treatment arms. Seventy one percent of patients treated with IV iron achieved an erythropoietic response, compared to 70% of patients treated with oral iron and 68% of patients treated with topical iron. This difference was not statistically significant. There were also no differences in the proportion of patients requiring red blood cell transfusions and changes in QoL.

### Example 5—Use of Topically Delivered Iron Supplementation for Treatment of Anemia in Inflammatory Bowel Disease

In this experiment, iron (in the form of ferrous sulfate) in formulations of the invention was tested for its ability to help treat anemia in inflammatory bowel disease (IBD) patients and compared to orally or intravenously delivered iron supplementation.

In vivo tests were performed as follows: human IBD patients with anemia were treated with either topical, oral or intravenous iron supplementation for 12 weeks. Trial design was built to assess this primary outcome: change in hemoglobin (Hb) from baseline to week 12. Treatment groups randomized as follows:
a. Oral iron supplement group (FeSulf containing 100 mg ferrous iron (II). Patients received one capsule b.i.d. for 12 wk)
b. Intravenous iron supplement group (ferric carboxymaltose (FeCarb) 50 mg ferric iron (III) per milliliter in water with individual patient dosage calculated according to the formula of Ganzoni)
c. Topical iron supplement group (1.67 g of topical formulation applied b.i.d.) Formulations detailed in Table 5 below

TABLE 5
Iron Formulation

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate | 6.00% |
| Total | 100.00% |

There was no statistically significant difference in the Hb change rate between the three treatment arms. The median Hb improved from 9.1 to 12.1 g/dL in the oral group, 8.7 to 12.3 g/dL in the IV group and 9.0 to 12.3 g/dL in the topical group.

### Example 6—Use of Topically Delivered Iron Supplementation to Improve Endurance after Training in Iron-Depleted, Nonanemic Women

In this experiment, iron (in the form of ferrous sulfate) in formulations of the invention was tested for its ability to help improve endurance after training in iron-depleted, nonanemic women and compared to orally delivered iron supplementation.

In vivo tests were performed as follows: human iron-depleted (serum ferritin <16 μg/l), nonanemic (Hb >12 g/dl) women (18-33 yr old) were treated with either topical or oral iron supplementation for 6 weeks. Subjects trained for 30 min/day, 5 days/wk at 75-85% of maximum heart rate for the final 4 wk of the study. Trial design was built to assess this primary outcome: change in 15 km time from baseline to week 12. Treatment groups randomized as follows:
a. Control group: oral placebo
b. Oral iron supplement group (100 mg of ferrous sulfate per day)
c. Topical iron supplement group (1.67 g of topical formulation applied daily) Formulations detailed in Table 6 below

TABLE 6
Iron Formulation

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate | 6.00% |
| Total | 100.00% |

All groups decreased 15-km time over the course of the study. The decrease in 15-km time was statistically significantly greater in the oral and topical supplementation groups vs the placebo control group. There was no statistical difference between the oral and transdermal.

### Example 7—Topical Iron Supplementation of Varying Particle Size to for Treatment of Iron Deficiency in Non-Hemodialysis-Dependent Patients with Chronic Kidney Disease

In this experiment, iron (in the form of ferrous sulfate) of various particle sizes in formulations of the invention was tested for its ability to treat iron deficiency in non-hemodialysis dependent patients with chronic kidney disease, and compared to orally delivered iron supplementation.

In vivo tests were performed as follows: Anemic human patients with progressive renal insufficiency were treated with either topical or oral iron supplementation. Trial design was built to assess these primary outcomes: hemoglobin response, and incidence of adverse events. Treatment groups randomized as follows:
d. Oral iron supplement group (ferrous sulphate 325 mg thrice daily for a total of 195 mg elemental iron daily)
e. Topical iron (Large Particle Size) supplement group (5.42 g of topical formulation applied tds) Formulation detailed below
f. Topical iron (Medium Particle Size) supplement group (5.42 g of topical formulation applied tds) Formulation detailed below
g. Topical iron (Small Particle Size) supplement group (5.42 g of topical formulation applied tds) Formulation detailed below

Group B - Iron Formulation (Large Particle Size)

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate (50 μm) | 6.00% |
| Total | 100.00% |

Group C - Iron Formulation (Medium Particle Size)

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate (1 μm) | 6.00% |
| Total | 100.00% |

Group D - Iron Formulation (Small Particle Size)

| Ingredient | Weight % |
|---|---|
| Phos 90G | 8.00% |
| Cetyl OH | 3.00% |
| Stearic Acid | 2.00% |
| Triacetin | 14.90% |
| lecithin | 4.00% |
| Almond oil | 12.00% |
| Cetiol Ultimate | 10.00% |
| Plu-Powder | 5.00% |
| Glacial Acetic Acid | 5.00% |
| Propanoic Acid | 2.00% |
| Cyclohexane | 5.00% |
| Tego 13-06 | 4.00% |
| Durosoft | 1.00% |
| HP beta-CD | 0.10% |
| Citric Acid | 2.00% |
| Vitamin C | 2.00% |
| Vitamin E | 2.00% |
| EDTA | 10.00% |
| Titanium Dioxide | 2.00% |
| Ferrous Sulfate (10 nm) | 6.00% |
| Total | 100.00% |

The proportion of subjects achieving a hemoglobin increase ≥1 g/dL at any time was 34.7% in the oral group, 52.1% in the topical group B (large particles), 64.7% in the topical group C (medium particles), and 78.1% in the topical group D (small particles). Treatment-related adverse events were significantly fewer with topical iron than with oral iron (4.3%, 5.0% and 3.8% vs 26.2%).

Aspects of the present specification may also be described as follows:

1. A formulation for transdermal delivery of iron or one or more iron containing compound through the skin of a subject, comprising
one or more iron containing compound in an amount of about 3.0% to about 40.0% w/w;
Phospholipon® 90G in an amount from 0.0% to 15.0% w/w; cetyl alcohol in an amount from 0.0% to 5.0% w/w; stearic acid in an amount from 0.0% to 5.0% w/w; triacetin in an amount from 0.0% to 40.0% w/w; lecithin in an amount from 0.0% to 10.0% w/w; almond oil in an amount from 0.0% to 15.0% w/w; Cetiol® Ultimate in an amount from 0.0% to 20.0% w/w; Poloxamer 407 in an amount of from 0.0% to 20.0% w/w; glacial acetic acid in an amount from 0.0% to 10.0% w/w; propanoic acid in an amount from 0.0% to 5.0% w/w; cyclohexane in an amount from 0.0% to 10.0% w/w; tego 13-06 in an amount from 0.0% to 10.0% w/w; Durosoft® PK-SG in an amount from 0.0% to 3.0% w/w; HP beta CD in an amount from 0.0% to 1.0% w/w; citric acid in an amount from 0.0% to 5.0% w/w; vitamin C in an amount from 0.0% to about 5.0% w/w; vitamin E in an amount from 0.0% to about 5.0% w/w; an iron chelator (e,g, EDTA) in an amount from 0.0% to about 20.0% w/w; titanium dioxide in an amount from 0.0% to about 5.0% w/w; and water.

2. A formulation according to claim 1, wherein said iron or one or more iron containing compound comprises ferrous sulfate.

3. A formulation for transdermal delivery of iron or one or more iron containing compound through the skin of a subject, comprising:
ferrous sulfate in an amount of about 3.0% to about 40.0% w/w;
and at least one of the following:
Phospholipon® 90G in an amount of about 5.0% w/w to about 15.0% w/w;
cetyl alcohol in an amount of about 1.0% w/w to about 5.0% w/w;
stearic acid in an amount of about 0.5% w/w to about 5.0% w/w;
triacetin in an amount of about 5.0% w/w to about 25.0% w/w;
lecithin in an amount of about 1.0% w/w to about 10.0% w/w;

37. A formulation according to any one of claims 1-9, comprising Tego 13-06 in an amount of about 4.0% w/w.

38. A formulation according to any one of claims 1-9, comprising Durosoft® PK-SG in an amount of about 1.0% w/w.

39. A formulation according to any one of claims 1-9, comprising HP beta CD in an amount of about 0.1% w/w.

40. A formulation according to any one of claims 1-9, comprising citric acid in an amount of about 2.0% w/w.

41. A formulation according to any one of claims 1-9, comprising vitamin C in an amount of about 2.0% w/w.

42. A formulation according to any one of claims 1-9, comprising vitamin E in an amount of about 2.0% w/w.

43. A formulation according to any one of claims 1-9, comprising EDTA in an amount of about 10.0% w/w.

44. A formulation according to any one of claims 1-9, comprising titanium dioxide in an amount of about 2.0% w/w.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability.
almond oil in an amount of about 3% w/w to about 15.0% w/w;
Cetiol® Ultimate in an amount of about 3.0% w/w to about 20.0% w/w;
Poloxamer 407 in an amount of about 2.0% w/w to about 20.0% w/w;
glacial acetic acid in an amount of about 2.0% w/w to about 10.0% w/w;
propanoic acid in an amount of about 1.0% w/w to about 5.0% w/w;
cyclohexane in an amount of about 2.0% w/w to about 10.0% w/w;
Tego 13-06 in an amount of about 2.0% w/w to about 10.0% w/w;
Durosoft® PK-SG in an amount of about 0.5% w/w to about 3.0% w/w;
HP beta CD in an amount up to about 1.0% w/w;
citric acid in an amount up to about 5.0% w/w;
vitamin C in an amount up to about 5.0% w/w;
vitamin E in an amount up to about 5.0% w/w;
EDTA in an amount up to about 20.0% w/w;
titanium dioxide in an amount up to about 5.0% w/w; and
H₂O to complete.

4. A formulation for transdermal delivery of iron or one or more iron containing compound through the skin of a subject, comprising:
ferrous sulfate in an amount of about 3.0% to about 40.0% w/w;
Phospholipon® 90G in an amount of about 5.0% w/w to about 15.0% w/w;
cetyl alcohol in an amount of about 1.0% w/w to about 5.0% w/w;
stearic acid in an amount of about 0.5% w/w to about 5.0% w/w;
triacetin in an amount of about 5.0% w/w to about 25.0% w/w;
lecithin in an amount of about 1.0% w/w to about 10.0% w/w;
almond oil in an amount of about 3% w/w to about 15.0% w/w;
Cetiol® Ultimate in an amount of about 3.0% w/w to about 20.0% w/w;
Poloxamer 407 in an amount of about 2.0% w/w to about 20.0% w/w;
glacial acetic acid in an amount of about 2.0% w/w to about 10.0% w/w;
propanoic acid in an amount of about 1.0% w/w to about 5.0% w/w;
cyclohexane in an amount of about 2.0% w/w to about 10.0% w/w;
Tego 13-06 in an amount of about 2.0% w/w to about 10.0% w/w;
Durosoft® PK-SG in an amount of about 0.5% w/w to about 3.0% w/w;
and optionally
HP beta CD in an amount up to about 1.0% w/w;
citric acid in an amount up to about 5.0% w/w;
vitamin C in an amount up to about 5.0% w/w;
vitamin E in an amount up to about 5.0% w/w;
EDTA in an amount up to about 20.0% w/w;
titanium dioxide in an amount up to about 5.0% w/w; and
H₂O to complete.

5. A formulation for transdermal delivery of iron or one or more iron containing compound through the skin of a subject, comprising:
ferrous sulfate in an amount of about 3.0% to about 40.0% w/w;
an iron chelator;
an antioxidant;
and optionally, one or more of the following:
Phospholipon® 90G in an amount of about 5.0% w/w to about 15.0% w/w;
cetyl alcohol in an amount of about 1.0% w/w to about 5.0% w/w;
stearic acid in an amount of about 0.5% w/w to about 5.0% w/w;
triacetin in an amount of about 5.0% w/w to about 25.0% w/w;
lecithin in an amount of about 1.0% w/w to about 10.0% w/w;
almond oil in an amount of about 3% w/w to about 15.0% w/w;
Cetiol® Ultimate in an amount of about 3.0% w/w to about 20.0% w/w;
Poloxamer 407 in an amount of about 2.0% w/w to about 20.0% w/w;
glacial acetic acid in an amount of about 2.0% w/w to about 10.0% w/w;
propanoic acid in an amount of about 1.0% w/w to about 5.0% w/w;
cyclohexane in an amount of about 2.0% w/w to about 10.0% w/w;
Tego 13-06 in an amount of about 2.0% w/w to about 10.0% w/w;
Durosoft® PK-SG in an amount of about 0.5% w/w to about 3.0% w/w;
HP beta CD in an amount up to about 1.0% w/w;
citric acid in an amount up to about 5.0% w/w;
titanium dioxide in an amount up to about 5.0% w/w; and
H₂O to complete.

6. A formulation according to any one of claims 1-5, comprising ferrous sulfate in an amount of about between about 3.0% w/w and about 15.0% w/w.

7. A formulation according to claim 6, comprising ferrous sulfate in an amount of about 6.0% w/w.

8. A formulation according to any one of claims 1-4, further comprising an iron chelator.

9. A formulation according to any one of claims 1-4, further comprising an antioxidant.

10. A method of treating an iron deficiency or related disorder in a subject in need thereof, the method comprising administering an effective amount of a formulation according to any one of claims 1-7, wherein said administration is effective to improve or treat the iron deficiency or related condition or disorder.

11. A method according to claim 10, wherein said iron deficiency or related disorder is anemia.

12. A method according to claim 10, wherein said iron deficiency or related disorder is geriatric anemia or an anemia associated with aging.

13. A method according to claim 10, wherein said iron deficiency or related disorder is anemia associated with sickle cell disease.

14. A method according to claim 10, wherein said iron deficiency or related disorder is an anemia associated with a bleeding disorder.

15. A method according to claim 10, wherein said iron deficiency or related disorder is an anemia associated with an iron deficiency.

16. A method according to claim 10, wherein said iron deficiency or related disorder is an anemia associated with a blood cancer.

17. A method according to claim 10, wherein said iron deficiency or related disorder is an anemia associated with a hematological disease or disorder.

18. A method according to claim 10, wherein said iron deficiency or related disorder is an anemia associated with a blood cancer.

19. A method according to claim 10, wherein said iron deficiency or related disorder is anemic associated with cancer.

20. A method according to claim 10, wherein said iron deficiency or related disorder is the supplementation of iron in non-hemodialysis patients with chronic kidney disease.

21. A method according to claim 10, wherein said iron deficiency or related disorder is progressive renal insufficiency.

22. A method according to claim 10, wherein said iron deficiency or related disorder is the supplementation of iron in a pregnant woman having or susceptible to having anemia.

23. A method according to claim 10, wherein said iron deficiency or related disorder is anemia associated with inflammatory bowel disease.

24. A method according to claim 10, wherein said iron deficiency or related disorder is to supplement iron in iron depletion associated with athletic training in non-anemic women.

25. A method according to claim 10, wherein said iron deficiency or related disorder is the supplementation of iron in anemic cancer patients also treated with darbepoetin alfa.

26. A formulation according to any one of claims 1-9, comprising Phospholipon® 90G in an amount of about 8.0% w/w.

27. A formulation according to any one of claims 1-9, comprising cetyl alcohol in an amount of about 3.0% w/w.

28. A formulation according to any one of claims 1-9, comprising stearic acid in an amount of about 2.0% w/w.

29. A formulation according to any one of claims 1-9, comprising triacetin in an amount of about 15% w/w.

30. A formulation according to any one of claims 1-9, comprising lecithin in an amount of about 4.0% w/w.

31. A formulation according to any one of claims 1-9, comprising almond oil in an amount of about 12% w/w.

32. A formulation according to any one of claims 1-9, comprising Cetiol® Ultimate in an amount of about 10.0% w/w.

33. A formulation according to any one of claims 1-9, comprising Poloxamer 407 in an amount of about 5.0% w/w.

34. A formulation according to any one of claims 1-9, comprising glacial acetic acid in an amount of about 5.0% w/w.

35. A formulation according to any one of claims 1-9, comprising propanoic acid in an amount of about 2.0% w/w.

36. A formulation according to any one of claims 1-9, comprising cyclohexane in an amount of about 5.0% w/w.

When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1.-20. (canceled)

21. A formulation for transdermal delivery of one or more iron-containing compounds through the skin of a subject, the formulation comprising:
an iron-containing compound;
an iron chelator;
an antioxidant; and
one or more of the following:
almond oil, Cetiol® Ultimate, cetyl alcohol, citric acid, cyclohexane, Durosoft, glacial acetic acid, H₂O, HP beta-CD, lecithin, Phospholipon® 90G, Poloxamer 407, propanoic acid, stearic acid, Tego 13-06, titanium dioxide, and triacetin.

22. The formulation of claim 21, wherein the iron chelator is one or more of:
ethylenediaminetetraacetic acid (EDTA), deferoxamine, 1,2-diethyl-3-hydroxypyridin-4-one (CP94), Desferol, Deferiprone and Deferasirox, succimer, trientine, Desferrithiocin, Clioquinol, 0-trensox, Tachpyr, Dexrazoxane, Triapine, Pyridoxal isonicotinoyl hydrazone, Di-2-pyridylketone thiosemicarbazone series, Flavan-3-ol, Curcumin, Apocynin, Kolaviron, Floranol, Baicalein, Baicalin, ligustrazine, Quercetin, Epigallocatechin gallate, Theaflavin, Phytic acid, and Genistein.

23. The formulation of claim 22, wherein the iron chelator is EDTA.

24. The formulation of claim 21, wherein the antioxidant is one or more of:
vitamin C, vitamin E, glutathione, superoxide dismutase, catalase, pNaKtide, Butylated hydroxytoluene, Butylated hydroxyanisole, tert-Butylhydroquinone, HP beta CD, resveratrol, retinol, coenzyme q10, niacinamide, polyphenols, flavenoids, beta-carotene, lutein, and lycopene.

25. The formulation of claim 24, wherein the antioxidant is vitamin C and/or vitamin E.

26. The formulation of claim 21, wherein the formulation comprises two or more of, three or more of, four or more of, five or more of, six or more of, seven or more of, eight or more of, nine or more of, ten or more of, eleven or more of, twelve or more of, thirteen or more of, fourteen or more of, fifteen or more of, or sixteen or more, or seventeen of:
almond oil, Cetiol® Ultimate, cetyl alcohol, citric acid, cyclohexane, Durosoft, glacial acetic acid, H₂O, HP beta-CD, lecithin, Phospholipon® 90G, Poloxamer 407, propanoic acid, stearic acid, Tego 13-06, titanium dioxide, and triacetin.

27. The formulation of claim 26, wherein when included in the formulation:
Phospholipon® 90G is present in an amount from about 5.0% w/w to about 15.0% w/w;
cetyl alcohol is present in an amount from about 1.0% w/w to about 5.0% w/w;
stearic acid is present in an amount from about 0.5% w/w to about 5.0% w/w;
triacetin is present in an amount from about 5.0% w/w to about 25.0% w/w;
lecithin is present in an amount from about 1.0% w/w to about 10.0% w/w;
almond oil is present in an amount from about 3% w/w to about 15.0% w/w;
Cetiol® Ultimate is present in an amount from about 3.0% w/w to about 20.0% w/w;
Poloxamer 407 is present in an amount from about 2.0% w/w to about 20.0% w/w;
glacial acetic acid is present in an amount from about 2.0% w/w to about 10.0% w/w;
propanoic acid is present in an amount from about 1.0% w/w to about 5.0% w/w;
cyclohexane is present in an amount from about 2.0% w/w to about 10.0% w/w;
Tego 13-06 is present in an amount from about 2.0% w/w to about 10.0% w/w;
Durosoft® PK-SG is present in an amount from about 0.5% w/w to about 3.0% w/w;
HP beta CD is present in an amount up to about 1.0% w/w;
citric acid is present in an amount up to about 5.0% w/w; and
titanium dioxide is present in an amount up to about 5.0% w/w.

28. The formulation of claim 21, wherein the iron-containing compound is selected from ferrous sulfate, sucrosomial iron, polysaccharide iron complex, ferrous fumarate, ferrous gluconate, ferric carboxymaltose, ferumoxytol, iron isomaltoside 1000, ferric gluconate, iron sucrose, and ferric pyrophosphate.

29. The formulation of claim 28, wherein the iron-containing compound comprises ferrous sulfate.

30. The formulation of claim 29, wherein the ferrous sulfate is present in the formulation in an amount from about 3.0% to about 40.0% w/w.

31. The formulation of claim 29, wherein the ferrous sulfate is present in the formulation in an amount from about 3.0% w/w to about 15.0% w/w.

32. The formulation of claim 29, wherein the ferrous sulfate is present in the formulation in an amount of about 6.0% w/w.

33. The formulation of claim 29, wherein the ferrous sulfate is present in particles narrower than about 10 nm.

34. The formulation of claim 29, wherein the ferrous sulfate is present in particles narrower than about 1 μm in diameter.

35. The formulation of claim 29, wherein the ferrous sulfate is present in particles of about 50 μm in diameter.

36. A method of treating an iron deficiency or related disorder in a subject in need thereof, the method comprising administering an effective amount of a formulation according to claim 21 to the subject,
wherein the effective amount of the formulation improves or treats the iron deficiency or the related disorder.

37. The method of claim 36, wherein the iron deficiency or related disorder is an anemia that is associated with: a sickle cell disease; a bleeding disorder; an iron deficiency; aging; a hematological disease or disorder; a blood cancer; a cancer; or with inflammatory bowel disease.

38. The method of claim 36, wherein the subject in need thereof is a non-hemodialysis patient with chronic kidney disease, a patient with progressive renal insufficiency, a pregnant woman having or susceptible to having anemia, or in a subject having iron depletion associated with athletic training.

39. A formulation for transdermal delivery of one or more iron containing compounds through the skin of a subject, comprising:
an iron containing compound comprising ferrous sulfate in an amount from about 3.0% to about 40.0% w/w;
Phospholipon® 90G in an amount from about 5.0% w/w to about 15.0% w/w;
cetyl alcohol in an amount from about 1.0% w/w to about 5.0% w/w;
stearic acid in an amount from about 0.5% w/w to about 5.0% w/w;
triacetin in an amount from about 5.0% w/w to about 25.0% w/w;
lecithin in an amount from about 1.0% w/w to about 10.0% w/w;
almond oil in an amount from about 3% w/w to about 15.0% w/w;
Cetiol® Ultimate in an amount from about 3.0% w/w to about 20.0% w/w;
Poloxamer 407 in an amount from about 2.0% w/w to about 20.0% w/w;
glacial acetic acid in an amount from about 2.0% w/w to about 10.0% w/w;
propanoic acid in an amount from about 1.0% w/w to about 5.0% w/w;
cyclohexane in an amount from about 2.0% w/w to about 10.0% w/w;
Tego 13-06 in an amount from about 2.0% w/w to about 10.0% w/w;
Durosoft® PK-SG in an amount from about 0.5% w/w to about 3.0% w/w; and
H₂O to complete; and
optionally comprising one or more of
HP beta CD in an amount up to about 1.0% w/w;
citric acid in an amount up to about 5.0% w/w;
vitamin C in an amount up to about 5.0% w/w;
vitamin E in an amount up to about 5.0% w/w;
EDTA in an amount up to about 20.0% w/w; and
titanium dioxide in an amount up to about 5.0% w/w.

40. A formulation for transdermal delivery of one or more iron containing compounds through the skin of a subject, comprising:
an iron containing compound comprising ferrous sulfate in an amount from about 3.0% to about 40.0% w/w; Phospholipon® 90G in an amount from about 0.0% to about 15.0% w/w; cetyl alcohol in an amount from about 0.0% to about 5.0% w/w; stearic acid in an amount from about 0.0% to about 5.0% w/w; triacetin in an amount from about 0.0% to about 40.0% w/w; lecithin in an amount from about 0.0% to about 10.0% w/w; almond oil in an amount from about 0.0% to about 15.0% w/w; Cetiol® Ultimate in an amount from about 0.0% to about 20.0% w/w; Poloxamer 407 in an amount of from about 0.0% to about 20.0% w/w; glacial acetic acid in an amount from about 0.0% to about 10.0% w/w; propanoic acid in an amount from about 0.0% to about 5.0% w/w; cyclohexane in an amount from about 0.0% to about 10.0% w/w; tego 13-06 in an amount from about 0.0% to about 10.0% w/w; Durosoft® PK-SG in an amount from about 0.0% to about 3.0% w/w; HP beta CD in an amount from about 0.0% to about 1.0% w/w; citric acid in an amount from about 0.0% to about 5.0% w/w; vitamin C in an amount from about 0.0% to about 5.0% w/w; vitamin E in an amount from about 0.0% to about 5.0% w/w; EDTA in an amount from about 0.0% to about 20.0% w/w; titanium dioxide in an amount from about 0.0% to about 5.0% w/w; and water to complete.
